SEQUENCE LISTING (1 sequence)

SEQ ID NO: 1 - protein, 7 aa, Homo sapiens
  PFTAIRE peptide
TFEAIRE

(19) United States
(12) Patent Application Publication
(10) Pub. No.: US 2023/0414701 A1
(43) Pub. Date: Dec. 28, 2023

(54) COMPOSITION FOR PREVENTING, AMELIORATING, OR TREATING COGNITIVE DYSFUNCTION DISEASE, COMPRISING PEPTIDE HAVING SYNAPTIC PLASTICITY CONTROL FUNCTION

(71) Applicant: UIF (University Industry Foundation), Yonsei University, Seoul (KR)

(72) Inventors: Young Soo KIM, Incheon (KR); Ill Hwan CHO, Cheonan-si, Chungcheongnam-do (KR); Tae Ho KO, Incheon (KR)

(73) Assignee: UIF (University Industry Foundation), Yonsei University, Seoul (KR)

(21) Appl. No.: 18/011,363

(22) PCT Filed: Jun. 18, 2021
(86) PCT No.: PCT/KR2021/007702

§ 371 Date: Aug. 17, 2023

(30) Foreign Application Priority Data
Jun. 18, 2020 (KR) 10-2020-0074556
Jun. 18, 2021 (KR) 10-2021-0079219

(51) Int. Cl. A61K 38/08; A61K 9/00; A61P 25/28

(52) U.S. Cl. CPC A61K 38/08; A61K 9/0056; A61P 25/28

(57) ABSTRACT

A method of preventing, ameliorating, or treating a cognitive dysfunction in a subject in need thereof, includes administering a pharmaceutical composition including a peptide represented by the amino acid sequence of SEQ ID NO: 1 as an active ingredient. Any one or more of the N-and C-termini of the peptide are modified and the modification is acetylation or amidation.

### CROSS-REFERENCE TO PRIOR APPLICATIONS

This Application is a National Stage Patent Application of PCT International Application No. PCT/KR2021/007702 (filed on Jun. 18, 2021), which claims priority to Korean Patent Application Nos. 10-2020-0074556 (filed on Jun. 18, 2020) and 10-2021-0079219 (filed on Jun. 18, 2021), which are all hereby incorporated by reference in their entirety.

### BACKGROUND

The present invention relates to a composition for preventing, alleviating or treating cognitive dysfunction, including a peptide capable of controlling synaptic plasticity, and more particularly, to a composition for preventing, alleviating or treating cognitive dysfunction, including a peptide represented by the amino acid sequence of SEQ ID NO: 1 as an active ingredient.

A synapse, which is the basic unit of signal transmission between nerve cells, consists of a presynapse, which is a site that releases a signal, i.e., a neurotransmitter, and a postsynapse, which is a site that receives a neurotransmitter. Various types of neurotransmitters are secreted from the presynapse, and in the human brain, neurons using glutamate (excitatory) and γ-aminobutyric acid (GABA; inhibitory) as neurotransmitters account for approximately 90%. The presynapse has different shapes and components depending on whether it is excitatory or inhibitory, an excitatory synapse mainly forms an extruded structure called a dendritic spine in a dendrite, and an inhibitory synapse is formed in a dendrite, soma, or a site where an axon starts without a protrusion. The dendritic spine is a very small structure with a length of more or less 3 μm, but includes all molecules that control postsynaptic signaling, or molecules necessary for synaptic plasticity in which the structure and properties are changed according to activity. Here, the dendritic spine includes a receptor, a synaptic adhesion protein, a signaling molecule, a scaffold protein, and a cytoskeleton. Particularly, glutamate receptors include an α-amino-3-hydroxy-5-methyl-4-isoxazolepropionic acid receptor (AMPAR), an N-methyl-D-aspartate receptor (NMDAR), and a metabotropic glutamate receptor (mGluR), and these receptors are being studied in connection with Alzheimer's disease. In excitatory synapses, the transmission of normal electrical signals occurs mainly through an AMPAR. When the synapse is strongly stimulated, as an NMDAR opens and calcium ions flow in, lower signaling proteins such as calcium/calmodulin-dependent protein kinase II (CaMK II) are activated and change to locate more AMPARs on the synaptic membrane. If this change continues, as the dendritic spine becomes bigger or is increased in number, the efficiency of synaptic transmission increases and is maintained for a long time (usually 1 hour or more), which is called long-term potentiation (LTP). Meanwhile, when synaptic stimulation is applied to a lesser degree than that in LTP induction, the phenomenon called long-term depression (LTD) occurs, and is induced according to different mechanisms by NMDAR or mGluR activation. In these two LTD types. a phosphatase and a protein inducing AMPAR endocytosis are activated to induce postsynaptic AMPAR removal and the reduction and loss of dendritic spines occur, resulting in attenuation of synaptic transmission. LTP and LTD are accepted as important mechanisms for learning and memory.

In this regard, synaptic degeneration, which is a pathophysiological characteristic of neurodegenerative diseases, is in the spotlight as a target for treatment of various neurodegenerative diseases, exhibiting cognitive dysfunction. For example, in the case of Alzheimer's disease, it has been reported that a decrease in the number of synapses following the accumulation of amyloid β is observed, and in many Alzheimer's disease mouse models, following amyloid β accumulation, a reduction in long-term potentiation of the hippocampus, a reduction in dendrite tissue, and synaptic loss were confirmed through two-photon imaging and electron microscopic imaging. In the case of Huntington's disease, it has been reported that synaptic loss is observed following the accumulation of mutated huntingtin protein, and is associated with cognitive dysfunction as the disease progresses, and also in the case of Parkinson's disease, in addition to motor and cognitive dysfunction, abnormalities in synaptic plasticity were observed (BRIC View, 2014-R16, 2014.10.14.).

Therefore, as a result of intensive studies for development of materials for preventing, alleviating or treating cognitive dysfunction by controlling synaptic plasticity, the present inventors confirmed that a peptide according to the present invention induces long-term potentiation in synapses of hippocampal tissue derived from an Alzheimer's disease mouse model to improve synaptic plasticity, and thus the present invention was completed.

### SUMMARY

Therefore, the present invention is directed to providing a composition for preventing, alleviating or treating cognitive dysfunction, including a peptide represented by the amino acid sequence of SEQ ID NO: 1 as an active ingredient.

However, technical problems to be solved in the present invention are not limited to the above-described problems, and other problems which are not described herein will be fully understood by those of ordinary skill in the art from the following descriptions.

To achieve the purposes of the present invention, the present invention provides a pharmaceutical composition for preventing or treating cognitive dysfunction, which includes a peptide represented by the amino acid sequence of SEQ ID NO: 1 as an active ingredient.

In addition, the present invention provides a food composition for preventing or alleviating cognitive dysfunction, which includes a peptide represented by the amino acid sequence of SEQ ID NO: 1 as an active ingredient.

In one embodiment of the present invention, the cognitive dysfunction may be a disease selected from the group consisting of Alzheimer's disease, cerebrovascular dementia, Pick's disease, Creutzfeldt-Jacob disease, dementia caused by a head injury, Parkinson's disease, amyotrophic lateral sclerosis, and Huntington's disease.

In another embodiment of the present invention, any one or more of the N- and C-termini of the peptide may be modified.

In still another embodiment of the present invention, the modification may be acetylation or amidation.

In yet another embodiment of the present invention, among such peptides, a peptide with modified N- and C-termini may be formed in a circular shape by bonding the N- and C-termini by an amide bond.

In yet another embodiment of the present invention, the peptide may increase the long-term potentiation (LTP) of a synapse.

In addition, the present invention provides a method of preventing or treating cognitive dysfunction, which includes administering a pharmaceutical composition including a peptide represented by the amino acid sequence of SEQ ID NO: 1 as an active ingredient into a subject.

In addition, the present invention provides a use of the pharmaceutical composition for preventing or treating cognitive dysfunction.

The present inventors experimentally confirmed that a peptide consisting of the amino acid sequence of SEQ ID NO: 1 directly binds to the CCNY protein, thereby increasing the long-term potentiation of synapses, and thus improving cognitive dysfunction, and thus the peptide according to the present invention can be effectively used for prevention, alleviation or treatment of various diseases exhibiting cognitive dysfunction.

### BRIEF DESCRIPTION OF THE DRAWINGS

FIG. 1 illustrates the process of synthesizing a peptide according to the present invention using a solid phase peptide synthesis method, which is an organic synthesis method.

FIG. 2A shows the structure of MIC-1 in which both termini of a peptide consisting of the amino acid of SEQ ID NO: 1 are modified according to the present invention.

FIG. 2B shows the structure of ring-shaped cyclo MIC-1 manufactured by bonding between the termini of MIC-1, both termini of which are modified.

FIG. 3A shows the result of confirming the synthesis of an unmodified peptide using RP-HPLC and ESI-MS methods.

FIG. 3B shows the result of confirming the synthesis of a modified peptide, in which an N-terminus is acetylated, using RP-HPLC and ESI-MS methods.

FIG. 3C shows the result of confirming the synthesis of a modified peptide, in which a C-terminus is amidated, using RP-HPLC and ESI-MS methods.

FIG. 3D shows the result of confirming the synthesis of a peptide, in which both termini are modified, using RP-HPLC and ESI-MS methods.

FIG. 3E shows the result of confirming the synthesis of a peptide, in which both termini are connected in a ring shape, using RP-HPLC and ESI-MS methods.

FIG. 4A shows the result of confirming the purification of an unmodified peptide using RP-HPLC and ESI-MS methods.

FIG. 4B shows the result of confirming the purification of an unmodified peptide using RP-HPLC and ESI-MS methods.

FIG. 4C shows the result of confirming the purification of a modified peptide, in which a C-terminus is amidated, using RP-HPLC and ESI-MS methods.

FIG. 4D shows the result of confirming the purification of a peptide, in which both termini are modified, using RP-HPLC and ESI-MS methods.

FIG. 4E shows the result of confirming the purification of a peptide, in which both termini are modified and connected in a circular shape, using RP-HPLC and ESI-MS methods.

FIG. 5 shows the result of ELISA to confirm the binding abilities of MIC-1 and cyclo MIC-1 to the CCNY protein.

FIG. 6 is the result of verifying a synaptic plasticity improving effect by treating brain hippocampal tissue section derived from an Alzheimer's disease animal model with MIC-1 and measuring fEPSP.

FIG. 7 is the result of analyzing an expression level of PSD-95 protein through Western blotting after treatment of Aβ42-treated cells with MIC-1 to see the role of MIC-1 in improving cognitive function.

FIG. 8 is the result of confirming an alternation value after a Y-maze behavioral test to determine the role of MIC-1 in improving cognitive function.

### DETAILED DESCRIPTION

As a result of intensive studies for development of materials for preventing, alleviating or treating cognitive dysfunction by controlling synaptic plasticity, the present inventors confirmed that a peptide according to the present invention induces long-term potentiation in synapses of hippocampal tissue derived from an Alzheimer's disease mouse model to improve synaptic plasticity, and thus the present invention was completed.

Hereinafter, the present invention will be described in detail.

The present invention provides a pharmaceutical composition for preventing or treating cognitive dysfunction, comprising a peptide represented by the amino acid sequence of SEQ ID NO: 1 as an active ingredient.

The term “prevention” used herein refers to all actions of inhibiting cognitive dysfunction or delaying the onset thereof by administration of the pharmaceutical composition according to the present invention.

The term “treatment” used herein refers to all actions involved in alleviating or beneficially changing symptoms of cognitive dysfunction by administration of the pharmaceutical composition according to the present invention.

In the present invention, the peptide represented by the amino acid sequence of SEQ ID NO: 1 may have modification(s) at one or more of N- and C-termini. Preferably, the modification is preferably acetylation or amidation, and more preferably, modification(s) of any one or more of the acetylation of an N-terminus and the amidation of a C-terminus.

In addition, in the present invention, a peptide represented by the amino acid sequence of SEQ ID NO: 1, in which both the N- and C-termini are modified, may be formed in a circular shape due to the amide bond between the N- and C-termini.

In the present invention, the “cognitive dysfunction” to be prevented or treated refers to a disease that can be caused by synaptic plasticity abnormalities, and may be alleviated or treated through the induction of long-term potentiation. Preferably, the cognitive dysfunction may be a disease selected from the group consisting of Alzheimer's disease, cerebrovascular dementia, Pick's disease, Creutzfeldt-Jacob disease, dementia caused by a head injury, Parkinson's disease, amyotrophic lateral sclerosis, and Huntington's disease, but the present invention is not limited thereto.

The term “synaptic plasticity” used herein is an important property of the nervous system in which the efficiency of synaptic transmission or the pattern of synaptic coupling consistently changes. Synaptic plasticity constitutes the basic processes of the brain's learning, memory, adaptation, and metabolic functions, and the foundation of high-level brain functions. Long-term potentiation is one of the most extensively studied forms of synaptic plasticity in the mammalian central nervous system. Long-term potentiation refers to a long-lasting increase in synaptic strength, and is regarded as a cytological mechanism of learning and memory.

In this regard, in the present invention, it was experimentally confirmed through specific examples that a peptide according to the present invention induces long-term potentiation in the hippocampal tissue and alleviates cognitive dysfunction.

Specifically, in one embodiment of the present invention, a peptide consisting of the amino acid sequence of SEQ ID NO: 1 corresponding to a partial sequence of Cdk14 was synthesized by an organic synthesis method (see Example 1), and to enhance the biological activity of the peptide, peptides with an amidated C-terminus, an acetylated N-terminus, and both modified termini, respectively, were synthesized and purified (see Example 2).

In another embodiment of the present invention, direct binding of the peptides according to the present invention to CCNY was confirmed through ELISA (see Example 4).

In still another embodiment of the present invention, as a result of treating hippocampal tissue extracted from an Alzheimer's disease animal model with the peptide with both modified termini, it was confirmed that long-term potentiation was induced to improve synaptic plasticity (see Example 5).

In yet another embodiment of the present invention, to identify the role of the peptide according to the present invention for improving cognitive dysfunction, as a result of Western blotting performed after the treatment of Aβ42-treated cells with the peptide, it was confirmed that the expression of PSD-95 protein, whose expression level is reduced by Aβ42 treatment, is increased, and through the Y-maze behavioral experiment, it was confirmed that the peptide of the present invention can restore memory and cognitive function (see Example 6).

The result of Example 6 showed that the peptide according to the present invention increases the long-term potentiation of synapses, confirming an improvement in cognitive dysfunction, and such a result proves that a composition including the peptide according to the present invention can be used for prevention, alleviation or treatment of cognitive dysfunction.

The pharmaceutical composition according to the present invention may include a peptide represented by the amino acid sequence of SEQ ID NO: 1 or a derivative thereof as an active ingredient, and further include a pharmaceutically acceptable carrier. The pharmaceutically acceptable carrier is generally used in formulation, and includes saline, distilled water, Ringer's solution, buffered saline, cyclodextrin, a dextrose solution, a maltodextrin solution, glycerol, ethanol, liposomes, etc., but the present invention is not limited thereto. If needed, the pharmaceutically composition may further include other conventional additives including an antioxidant, a buffer, etc. In addition, by additionally adding a diluent, a dispersant, a surfactant, a binder or a lubricant, the pharmaceutical composition may be formulated as an injectable form such as an aqueous solution, an emulsion or a suspension, a pill, a capsule, a granule or a tablet. Suitable pharmaceutically acceptable carriers and their formulations may be formulated according to each ingredient using a method disclosed in Remington's Pharmaceutical Science. The pharmaceutical composition of the present invention is not limited to dosage form, and thus may be formulated as an injection, an inhalant, or a dermal preparation for external use.

The pharmaceutical composition of the present invention may be administered orally or non-orally (e.g., intravenously, subcutaneously, percutaneously, nasally or intratracheally) according to a desired method, and a dose of the pharmaceutical composition of the present invention may be selected according to a patient's condition and body weight, severity of a disease, dosage form, an administration route and duration by those of ordinary skill in the art.

The composition according to the present invention is administered at a pharmaceutically effective amount. In the present invention, the “pharmaceutically effective amount” used herein refers to an amount sufficient for treating a disease at a reasonable benefit/risk ratio applicable for medical treatment, and an effective dosage may be determined by parameters including a type of a patient's disease, severity, drug activity, sensitivity to a drug, administration time, an administration route and an excretion rate, the duration of treatment and drugs simultaneously used, and other parameters well known in the medical field. The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without a side effect, and such a dose may be easily determined by one of ordinary skill in the art.

In another aspect of the present invention, the present invention provides a food composition for preventing or alleviating cognitive dysfunction, including a peptide represented by the amino acid sequence of SEQ ID NO: 1 or a derivative thereof as an active ingredient.

The term “alleviation” used herein refers to all types of actions that at least reduce parameters related to a condition to be treated, for example, the severity of a symptom, and may be used with or without a drug for treatment before or after the onset of a corresponding disease.

The term “food composition” used herein includes one or more of a carrier, a diluent, an excipient and an additive and is formulated in one selected from the group consisting of tablets, pills, a powder, granules, capsules, and a liquid. Foods that can be added to the present invention may include various foods, powders, granules, tablets, capsules, syrups, beverages, gums, teas, vitamin complexes, and health functional foods. As an additive further included in the present invention, one or more types of ingredients selected from the group consisting of natural carbohydrates, sweeteners, nutrients, vitamins, minerals (electrolytes), flavoring agents (synthetic flavoring agents, natural flavoring agents, etc.), coloring agents, fillers, pectic acid and a salt thereof, alginic acid and a salt thereof, organic acids, protective colloid thickening agents, pH modifiers, stabilizers, preservatives, antioxidants, glycerin, alcohols, carbonating agents, and fruit flesh may be used. Examples of the above-mentioned natural carbohydrates include conventional sugars, for example, monosaccharides such as glucose, fructose, etc.; disaccharides such as maltose, sucrose, etc.; and polysaccharides such as dextrin, cyclodextrin, etc., and sugar alcohols such as xylitol, sorbitol, erythritol, etc. As the sweeteners, natural sweeteners [thaumatin, stevia extract (e.g., rebaudioside A, glycyrrhizin, etc.)] and synthetic sweeteners (saccharin, aspartame, etc.) may be advantageously used. In addition to the above ingredients, the composition according to the present invention may contain a variety of nutrients, vitamins, minerals (electrolytes), flavoring agents including synthetic and natural flavoring agents, coloring agents and fillers, pectic acid and a salt thereof, alginic acid and a salt thereof, an organic acid, protective colloid thickening agents, pH modifiers, stabilizers, preservatives, glycerin, alcohols, or carbonating agents used in carbonated beverages. In addition, the composition according to the present invention may contain flesh for preparing natural fruit juices and vegetable juices. Such ingredients may be used independently or in combination. Specific examples of the carriers, excipients, diluents and additives may include, but are not limited to, one or more selected from the group consisting of lactose, dextrose, sucrose, sorbitol, mannitol, erythritol, starch, acacia gum, calcium phosphate, alginate, gelatin, calcium phosphate, calcium silicate, microcrystalline cellulose, polyvinylpyrrolidone, cellulose, methyl cellulose, water, sugar syrup, methyl hydroxybenzoate, propyl hydroxybenzoate, talc, magnesium stearate, and mineral oil.

In still another aspect of the present invention, the present invention provides a method of preventing or treating cognitive dysfunction, including administering a pharmaceutical composition for preventing or treating cognitive dysfunction, including a peptide represented by the amino acid sequence of SEQ ID NO: 1 as an active ingredient, into a subject.

The term “subject” used herein refers to a target in need of treatment, and more specifically, a mammal such as a human or a non-human primate, a mouse, a rat, a dog, a cat, a horse, or a cow.

In yet another aspect of the present invention, the present invention provides a use of the pharmaceutical composition for preventing or treating cognitive dysfunction.

Hereinafter, to help in understanding the present invention, exemplary examples will be suggested. However, the following examples are merely provided to more easily understand the present invention, and not to limit the present invention.

### EXAMPLES

### Example 1. Peptide Synthesis

As a result of continuous studies to discover a material for controlling synaptic plasticity in neurodegenerative diseases exhibiting cognitive dysfunction, such as Alzheimer's disease, the present inventors anticipated that the PFTAIRE sequence, which is an important sequence in the Cdk14 kinase protein, binds to the CCNY protein to control synaptic plasticity, and tried to synthesize the target sequence peptide and verify its effect.

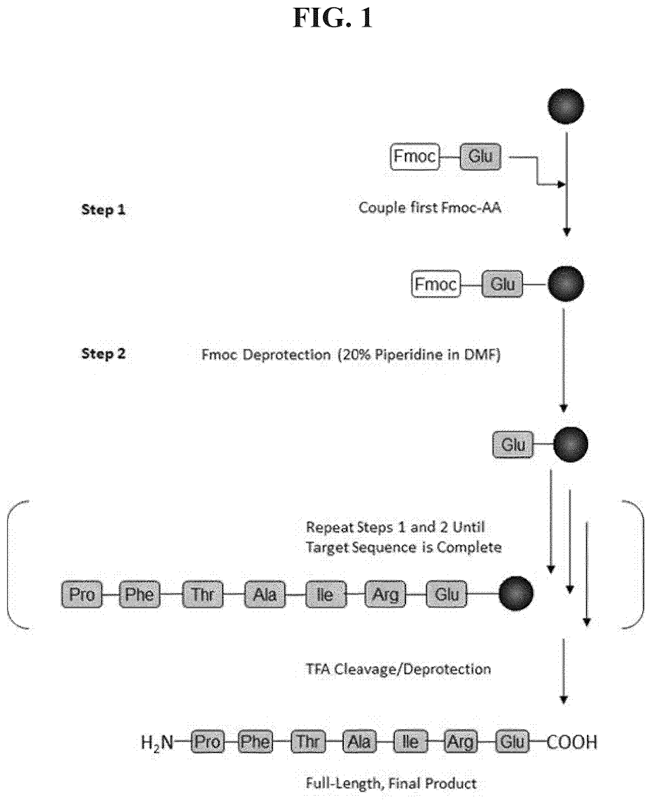

First, to synthesize the peptide, a solid phase peptide synthesis method, which is an organic synthesis method known in the art, was used, and the peptide synthesis process is illustrated in FIG. 1. Specifically, amino acids were bound to the TentaGel R Resin to synthesize a target peptide material, and particularly, after the first amino acid was bound to the TentaGel R Resin, an Fmoc protecting group at the N-terminus was removed with 20% piperidine, and subsequent amino acids were bound thereto. This process was repeated until the last amino acid was bound. Subsequently, after the synthesis of the target peptide material, the synthesized peptide was separated from the TentaGel R Resin and other side chain protecting groups, lyophilized, and stored. In addition, the synthesized peptide was named MIC-1.

### Example 2. Peptide Modification

To increase the activity of the peptide synthesized in Example 1, the present inventors devised a method of modifying the C- and N-termini of the peptide. Therefore, peptides each having both termini modified together, or both termini modified into a ring shape were prepared by methods as below, respectively.

### 2-1. Synthesis of C-Terminus-Modified Peptide

First, to synthesize a peptide in which a C-terminus was amidated, a target protein was synthesized by binding amino acids to PL-AMS Resin using the solid phase peptide synthesis method described in Example 1. More specifically, a C-terminus was amidated by binding a linker to the PL-AMS Resin. The amino acid binding process was performed in the same manner as in Example 1. That is, after binding of the first amino acid, an Fmoc protecting group at the N-terminus was removed with 20% piperidine, and then subsequent amino acids were bound. This process was repeated until the final amino acid was bound. After the synthesis of the target peptide, the synthesized peptide was separated from the PL-AMS Resin and other side chain protecting groups, lyophilized, and stored.

### 2-2. Synthesis of N-terminus-Modified Peptide

Afterward, to synthesize a peptide with an acetylated N-terminus, a target peptide was synthesized by binding amino acids until the final one using the solid phase peptide synthesis method described in Example 1. Subsequently, acetic anhydride and N,N-diisopropylethylamine were added and reacted for 2 hours to acetylate the final N terminus of the peptide material. Afterward, synthesized and N-terminus-modified peptides were separated from the TentaGel R Resin and other side chain protecting groups, lyophilized, and stored.

### 2-3. Synthesis of Peptide Modified at Both Termini

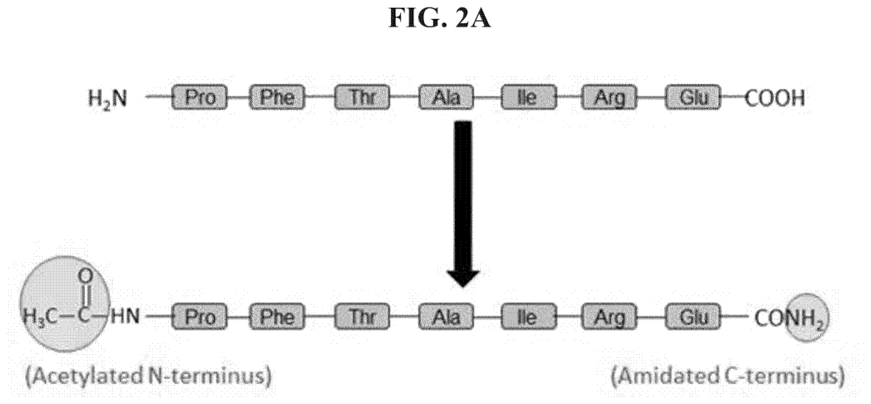

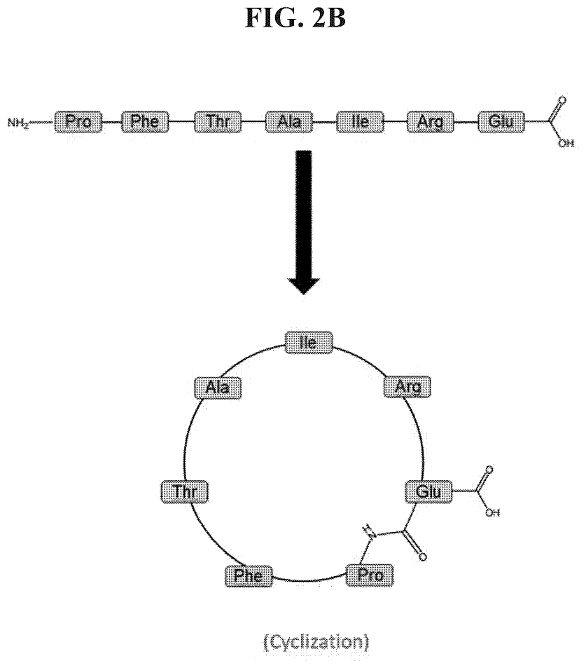

To synthesize a peptide in which the C- and N-termini were simultaneously modified at the same time, the present inventors synthesized a peptide by combining the methods described in Examples 2-1 and 2-2. More specifically, a C-terminus was amidated by binding a linker to PL-AMS Resin. After binding the first amino acid, an Fmoc protecting group at the N-terminus was removed with 20% piperidine, and then subsequent amino acids were bound. This process was repeated until the last amino acid was bound. Subsequently, acetic anhydride and N,N-diisopropylethylamine were added and reacted for 2 hours to acetylate the final N terminus of the peptide material. Afterward, a peptide modified at both termini was separated from the PL-AMS Resin and other side chain protecting groups, lyophilized, and stored. The sequence and structure of the peptide according to the present invention, in which both termini are modified, are shown in FIG. 2A, and this peptide was named “MIC-1.”

### 2-4. Synthesis of Peptide in Which Both Termini are Modified into Ring Shape

Ring-shaped MIC-1 was made by binding an amine group at the N-terminus and a side chain group at a glutamate of MIC-1 obtained in Example 2-3 through an amide bond. The structure of the peptide in which both termini of the MIC-1 obtained as described above are modified into a ring shape is shown in FIG. 2B, and this peptide was named “cyclo MIC-1.”

### Example 3. Peptide synthesis verification and purification

To confirm whether each of the peptides synthesized according to Examples 1 and 2 was well synthesized, the present inventors were analyzed though analysis methods, for example, RP-HPLC and ESI-MS.

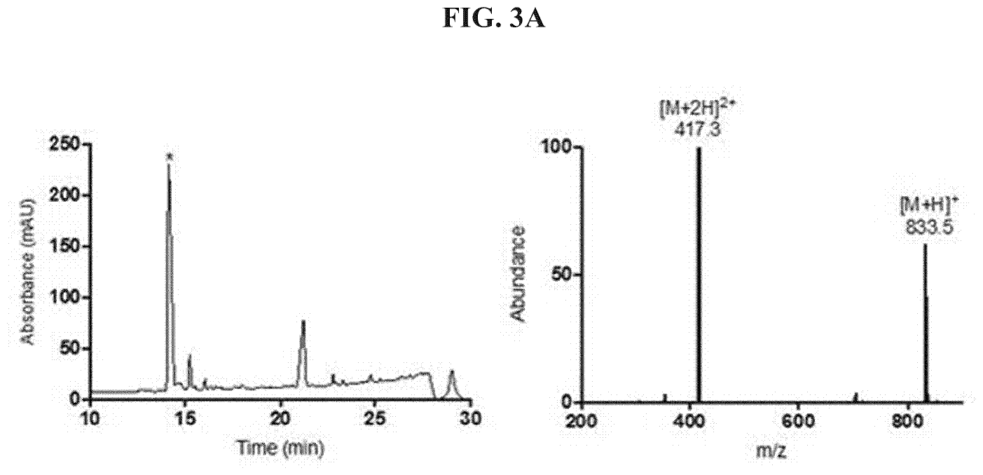

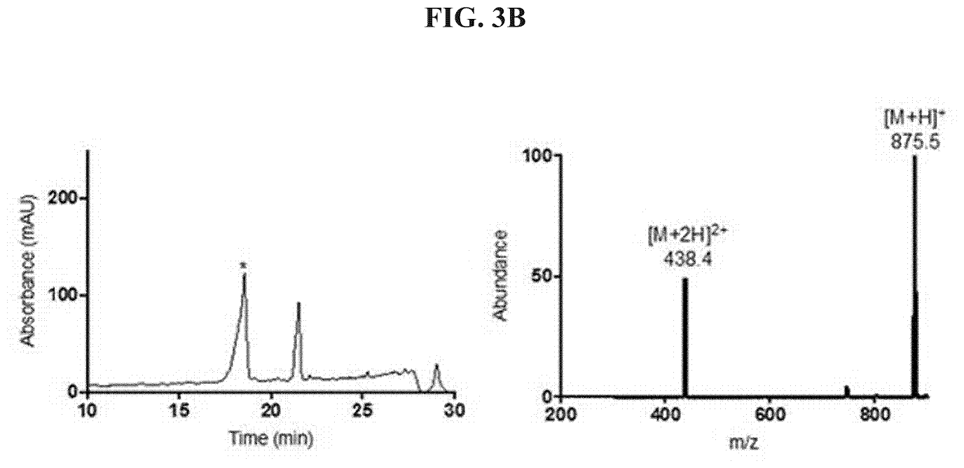

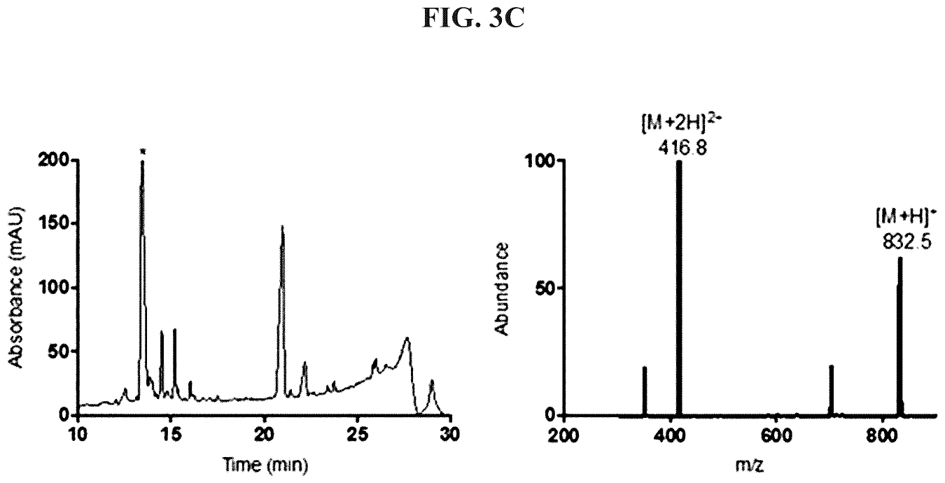

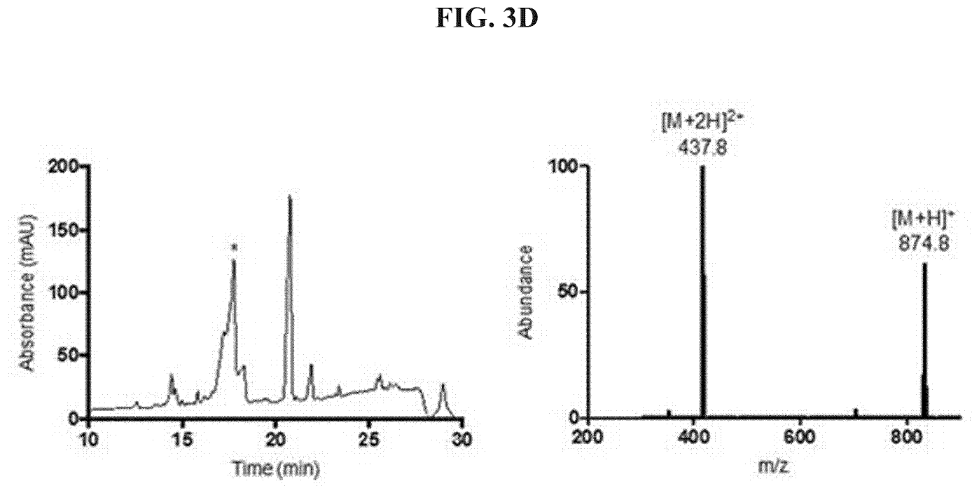

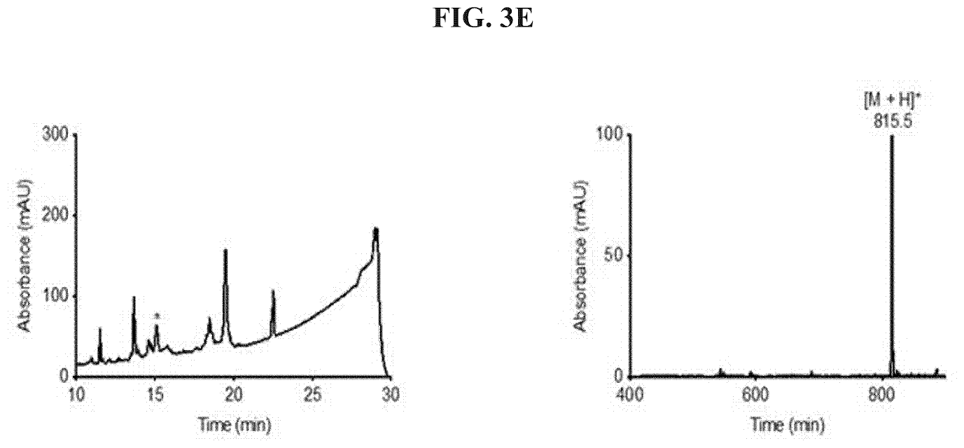

Specifically, a flow rate of 0.8 mL/min was applied to a Kintex 5u C18 (150 x 4.6mm) column, and then synthesized materials were analyzed. As a result, as shown in FIGS. 3A to 3E, it was confirmed whether a peptide with unmodified termini, a peptide with an acetylated N-terminus, a peptide with an amidated C-terminus, a peptide with both modified termini, and a peptide in which both modified termini are connected in a ring shape are well synthesized.

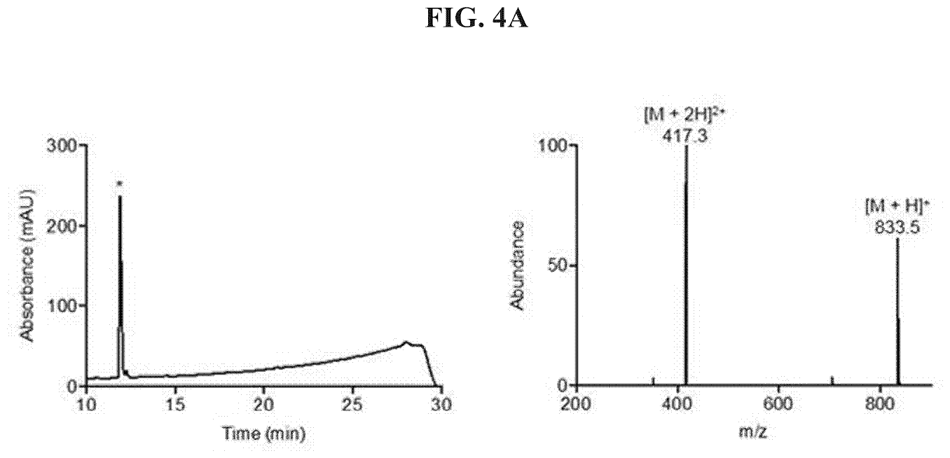

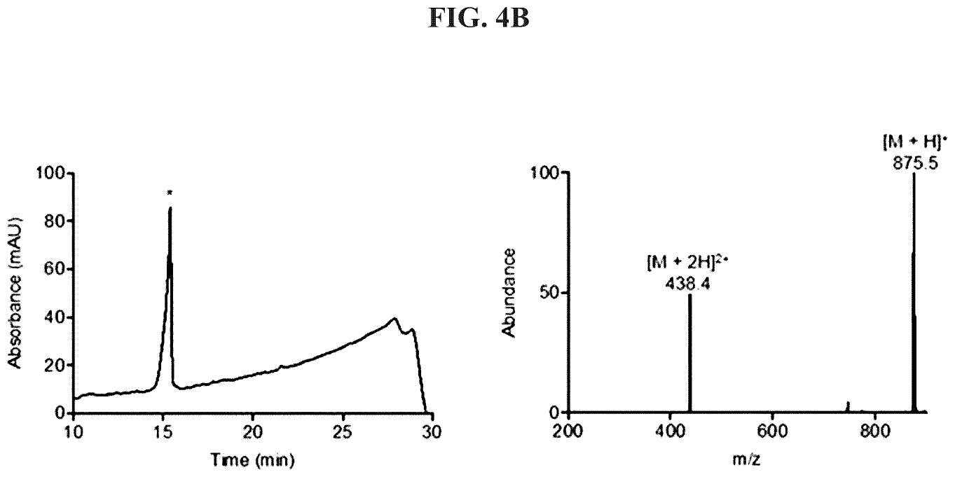

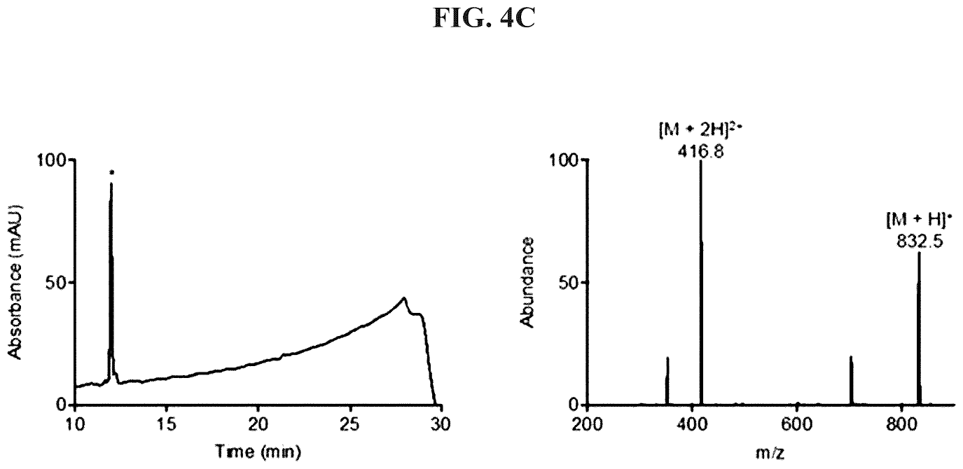

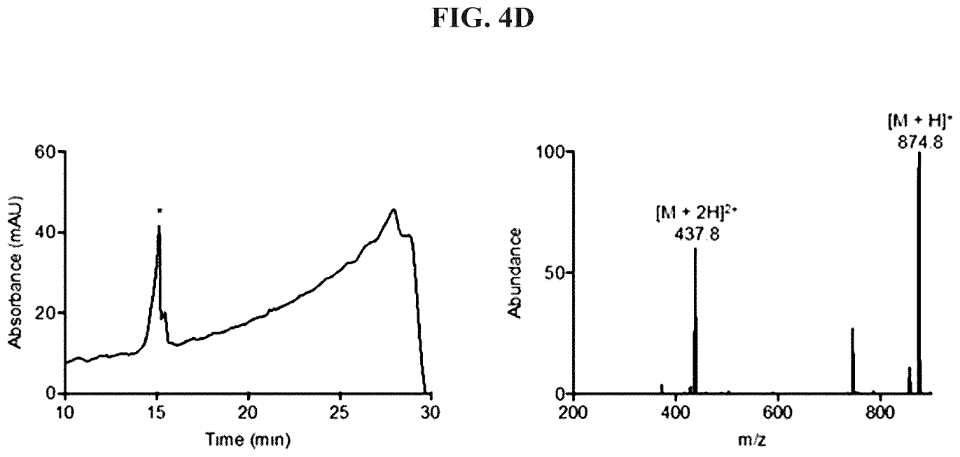

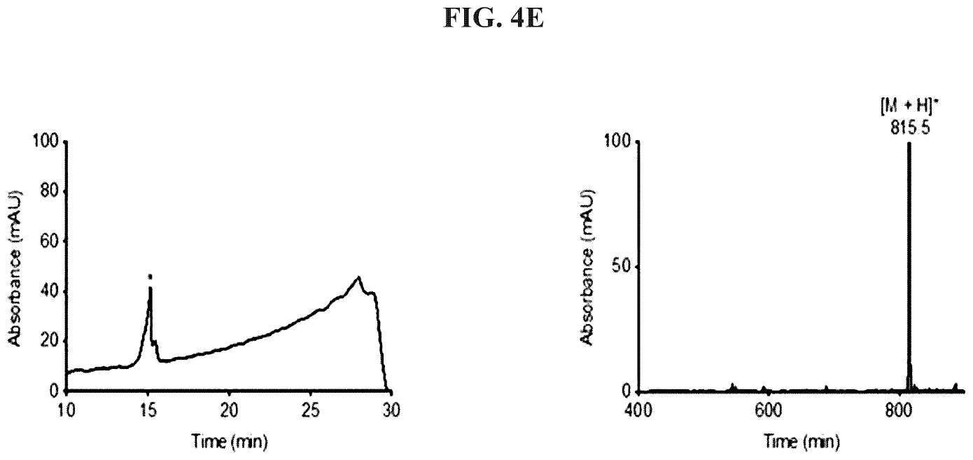

Moreover, the present inventors applied a flow rate of 6.0 mL/min to a Jupiter 10u C18 3029 (250×21.20 mm) column using two kinds of mobile phases, A (0.1% TFA in DW) and B (0.09% TFA in acetonitrile), and then the synthesized peptides were purified. Afterward, after applying a flow rate of 0.8 mL/min to a Kintex 5u C18 (150×4.6 mm) column, the synthesized materials were analyzed. As a result, as shown in FIGS. 4A to 4E, it was confirmed that four types of the peptides are all purified well.

### Example 4. Analysis of Binding Capacity of Peptides of the Present Invention to CCNY

To examine which of MIC-1 and cyclo MIC-1 according to the present invention easily binds to CCNY, the present inventors conducted an experiment using ELISA. To this end, CCNY was immobilized on a plate, MIC-1 and cyclo MIC-1 were treated at 10, 100 or 1000μg/mL, biotin was bound to the MIC-1 and cyclo MIC-1, and then the optical density of HRP was measured after treating streptavidin-HRP.

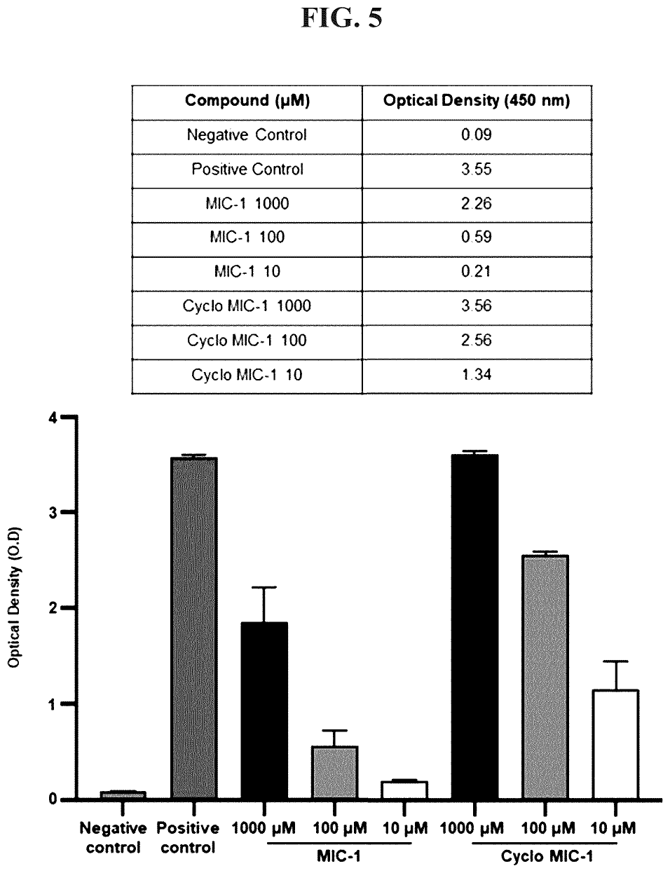

As a result, as shown in FIG. 5, it was confirmed that both MIC-1 and cyclo MIC-1 can remarkably bind to the CCNY protein, and particularly, when cyclo MIC-1 was treated at 1000 μg/mL, it was found that it exhibits a binding capacity that is as strong as the CCNY target antibody used as a positive control.

### Example 5. Verification of Synaptic Plasticity Controlling Effect of MIC-1

The present inventors attempted to verify the synaptic plasticity controlling effect of MIC-1, which is a peptide in which both termini were modified, synthesized and purified through Examples 1 to 3. To this end, they investigated whether long-term potentiation mediated by MIC-1 is induced in an Alzheimer's disease animal model.

Specifically, the brain was extracted from an APP/PS1 Alzheimer's disease animal model, and then transferred to a 4° C. sucrose-artificial cerebrospinal fluid solution (195.5 sucrose, 2.5 KC1, 1 NaH₂PO₄, 32.5 NaHCO₃, 11 glucose, 2 NaPyruvate, 1 NaL-ascorbate, 5 MgSO₄, 0.5 CaCl₂ (95% O₂/5% CO₂). Subsequently, brain tissue was cut into coronal sections (400 μm) using a microtome, a hippocampal tissue section was transferred to a 35° C. artificial cerebrospinal fluid solution (128.5 NaCl₂, 2.5 KCl, 1 NaH₂PO₄, 21.7 NaHCO₃, 11 glucose, 2 NaPyruvate, 1 NaL-ascorbate, 5 MgSO₄, 1 CaCl₂ (95% O₂/5% CO₂)) and incubated for 30 minutes to restore the function of the tissue. Subsequently, an experiment was performed for 1 hour and 40 minutes while flowing MIC-1 according to the present invention at a concentration of 1 μM from the beginning until the end of the experiment using gravity, and then the amplitude of field excitatory postsynaptic potential (fEPSP) was measured.

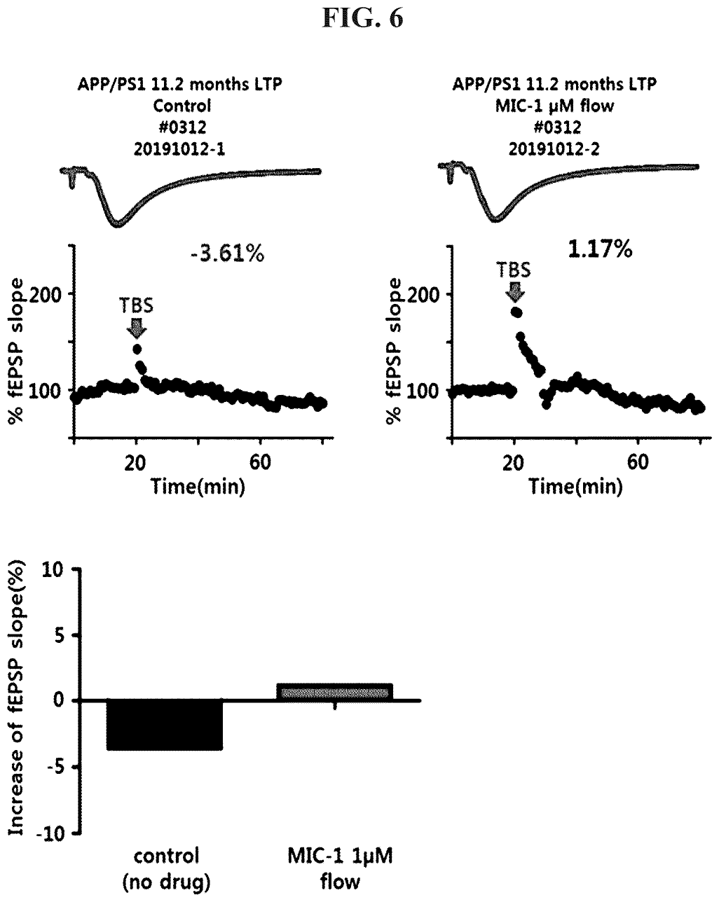

As a result, as shown in FIG. 6, it was shown that, compared to the treatment with a control peptide material, when MIC-1 according to the present invention is treated, the fEPSP amplitude increases. According to this, it was confirmed that long-term potentiation, which is one form of synaptic plasticity, was induced.

### Example 6. Confirmation of Role of MIC-1 in Improvement in Cognitive Function

### 6-1. Confirmation of Change in Expression Level of PSD-95 Protein by MIC-1 Treatment

To identify what role MIC-1 plays in improving the cognitive function of MIC-1 according to the present invention, the present inventors, as shown in Example 5, an experiment was performed as follows. Specifically, the change in expression level of the PSD-95 protein known to play an important role in cognitive function was analyzed through Western blotting using a 12% acrylamide gel. For example, it is known that, when cells are treated with Aβ42, compared to normal cells, the amount of PSD-95 protein decreases and cognitive function decreases, resulting in deterioration of memory.

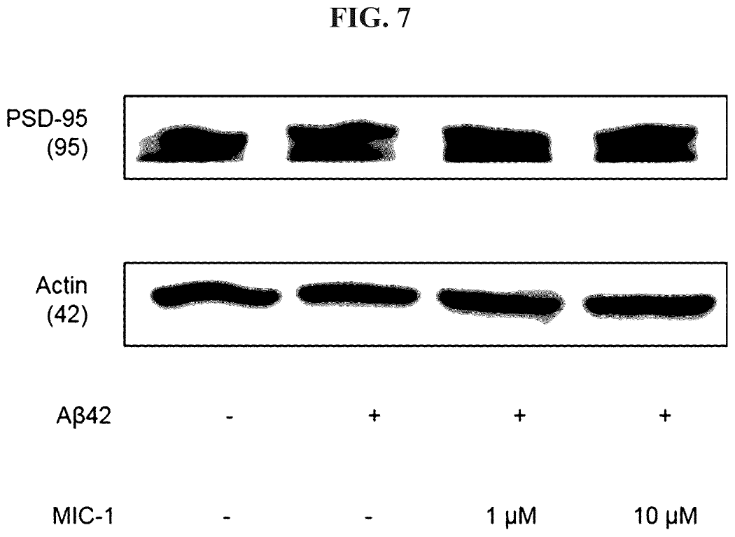

As a result of the experiment, as shown in FIG. 7, it was confirmed that, when Aβ42-treated cells are treated with MIC-1, cognitive function is increased as the level of the PSD-95 protein is increased. Through this, it was found that MIC-1 plays an important role in cognitive function.

### 6-2. Confirmation of Memory Improving Capacity of MIC-1

In Example 6-1, it was confirmed that MIC-1 can enhance the expression level of the PSD-95 protein known to be associated with cognitive function, and based on this, a behavioral experiment for mouse models was designed. Specifically, among ICR mouse models, 7-week-old male mouse models were used and divided into a control and two experimental groups (scopolamine-treated group and group administered MIC-1 and then treated with scopolamine) to perform the experiment.

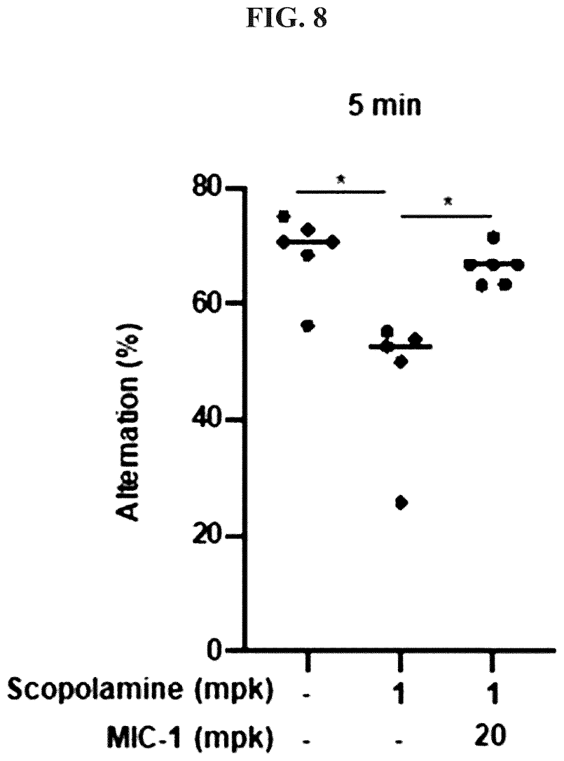

MIC-1 was administered into a mouse twice via intravenous administration at 20 mpK for one week. Rodents have a habit of exploring a new area, and when memory is temporarily reduced with scopolamine, they keep selecting the areas that they have already passed through and passing through these areas in the Y-maze, thereby drastically decreasing alternation. Therefore, to induce such memory loss, 30 minutes before the Y-maze behavioral experiment, scopolamine was intraperitoneally administered at 1 mpK to the experimental groups, resulting in memory deterioration. As a result of measuring alternation through the Y-maze behavioral experiment for each group, as shown in FIG. 8, it was confirmed that, in the group treated with MIC-1 and then scopolamine, it was confirmed that the memory is improved up to the control level.

From the above results, the present inventors confirmed through a specific behavioral experiment that the MIC-1 of the present invention affects memory recovery and can restore cognitive function.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as being illustrative and not restrictive in any aspect.

The present invention relates to a composition for preventing, alleviating or treating cognitive dysfunction, including a peptide for controlling synaptic plasticity, and it was experimentally confirmed that the peptide consisting of the amino acid sequence of SEQ ID NO: 1 of the present invention directly binds to the CCNY protein, resulting in increasing the long-term potentiation of synapses and thus improving cognitive dysfunction. Therefore, the peptide according to the present invention may be effectively used for the prevention, alleviation or treatment of various diseases exhibiting cognitive dysfunction.

1. A method of preventing or treating cognitive dysfunction in a subject in need thereof, comprising:
administering a pharmaceutical composition comprising a peptide represented by the amino acid sequence of SEQ ID NO: 1 as an active ingredient.

2. The method of claim 1, wherein the cognitive dysfunction is a disease selected from the group consisting of Alzheimer's disease, cerebrovascular dementia, Pick's disease, Creutzfeldt-Jacob disease, dementia caused by a head injury, Parkinson's disease, amyotrophic lateral sclerosis, and Huntington's disease.

3. The method of claim 1, wherein any one or more of the N- and C-termini of the peptide are modified.

4. The method of claim 3, wherein the modification is acetylation or amidation.

5. The method of claim 3, wherein a peptide with the modified N- and C-termini among peptides is formed in a cyclic shape due to bonding between the N- and C-termini by an amide bond.

6. The method of claim 1, wherein the peptide increases long-term potentiation (LTP).

7. A food composition for preventing or improving cognitive dysfunction, comprising a peptide represented by the amino acid sequence of SEQ ID NO: 1 as an active ingredient.

8. The food composition of claim 7, wherein the cognitive dysfunction is a disease selected from the group consisting of Alzheimer's disease, cerebrovascular dementia, Pick's disease, Creutzfeldt-Jacob disease, dementia caused by a head injury, Parkinson's disease, amyotrophic lateral sclerosis, and Huntington's disease.

9. The food composition of claim 7, wherein any one or more of the N- and C-termini of the peptide is modified.

10. The food composition of claim 9, wherein the modification is acetylation or amidation.

11. The food composition of claim 9, wherein a peptide with the modified N- and C-termini among peptides is formed in a cyclic shape due to bonding between the N- and C-termini by an amide bond.

12. The food composition of claim 7, wherein the peptide increases long-term potentiation (LTP).

13. (canceled)

14. (canceled)
